(19) United States
(12) Patent Application Publication
(10) Pub. No.: US 2025/0269080 A1
(43) Pub. Date: Aug. 28, 2025

(54) UV ENABLED FINS ENCAPSULATION SYSTEM

(71) Applicant: UV AMERICAN TECHNOLOGY, LLC, Bethesda, MD (US)

(72) Inventors: Ilyas HAMIDZAI, Bethesda, MD (US); Gary DAVIDSON, Bethesda, MD (US)

(21) Appl. No.: 19/201,562

(22) Filed: May 7, 2025

Related U.S. Application Data
(63) Continuation of application No. PCT/US2023/079214, filed on Nov. 9, 2023.
(60) Provisional application No. 63/423,993, filed on Nov. 9, 2022.

(51) Int. Cl. A61L 9/20

(52) U.S. Cl. CPC A61L 9/20; A61L 2209/12; A61L 2209/14; A61L 2209/16

(57) ABSTRACT

A germicidal UVC LED array includes a fin design that uses a heatsink to dissipate the high-powered UVC LEDs generated heat, redirect the air to increase or decrease the air pressure providing pressure stabilization, and redirect the air to a channel to expose it to the maximum amount of irradiation from the UVC LEDs. This design creates air vortexes to expose the pathogens to radiation for a longer period of time or to direct the pathogens to higher light-intensity regions illuminated by the LEDs. The fin arrangement with the heatsink can be arranged at any angle. It can be placed around the edge of the duct system to provide a frame.

### CROSS-REFERENCES TO RELATED APPLICATIONS

Priority is claimed from U.S. provisional patent application No. 63/423,993 filed Nov. 9, 2022, incorporated by reference for all purposes.

### STATEMENT REGARDING FEDERALLY SPONSORED RESEARCH OR DEVELOPMENT

None.

### BACKGROUND & SUMMARY

Recent increases in outbreaks of severe airborne viral infectious diseases that attack respiratory systems caused by viruses have led to epidemic and pandemic spread with little to no immunity. In particular, Severe Acute Respiratory Syndrome Coronavirus 2 (SARS COV-2) was declared an international pandemic in late 2019. The single-strand enveloped RNA virus belongs to the family of Coronaviridae, and the SARS COV-2 is unlikely to disappear but instead become part of the repertoire of repertory viruses that infect humans regularly. Furthermore, precautions such as social distancing indoors that were taken during the pandemic to avoid the spread of SARS COV-2 appear to have had the effect of protecting large parts of the population from other viral respiratory diseases such as influenza and Respiratory Syncytial Virus Infection (RSV), which are now spreading to those with low or no built-up immunity with often devastating effects.

Besides continuous use of Personal Protection Equipment (PPE), sanitizing air circulation systems is recommended by healthcare professionals as prevention measures for infectious diseases. Ultraviolet light has been demonstrated to deactivate viruses. See e.g., Raeiszadeh et al, “A Critical Review on Ultraviolet Disinfection Systems against COVID-19 Outbreak: Applicability, Validation, and Safety Considerations”, ACS Photonics. 2020 Oct. 14: acsphotonics.0c01245 published online 2020 Oct. 14. doi: 10.1021/acsphotonics.0c01245. Our previous technology disclosed in U.S. Pat. No. 10,946,321 (incorporated herein by reference) took advantage of such UV effects to provide a fin-based UV structure added to a conventional air filter.

One of the challenges of using UV light to deactivate viruses relates to providing sufficient UV light intensity to be efficacious in deactivating pathogens. Various UV light sources have been explored. While UV light-emitting diodes have many advantages over other sources, such as mercury vapor and excimer sources, a major disadvantage of LEDs is heat dissipation. Thus, thermal management of the luminaires containing many LEDs must be included in the design of the luminaire. For example, if it is desired to provide an Upper Room UVGI using LEDs that produce 5 watts of UV-C output, one would need 250 watts, e.g., 250 1-W LEDS, with assumed efficiency of 2% (half of light lost in keeping radiation in upper room). This would create a significant thermal management problem for the luminaire design to keep LED junctions at a reasonable temperature. Note that the radiant flux, life, and reliability of LEDs decrease significantly with increasing temperature. Bergman, “Germicidal UV Sources and Systems” (Wiley 26 Jan. 2021) doi.org/10.1111/php.13387.

Heat sinks are well-known structures used to dissipate heat generated by electronic components. See Ahmed et al, “Optimization of thermal design of heat sinks: A review”, International Journal of Heat and Mass Transfer Volume 118, March 2018, Pages 129-153, doi.org/10.1016/j.ijheatmasstransfer.2017.10.099. Heat sinks are conventionally built into substrates of UVC LEDs to dissipate heat generated by the LEDs. But as LED intensity increases, heat dissipation can still become a problem, as described above.

The technology herein provides a unique fin-based filtration system that implements effective methods and techniques to eliminate the activation of airborne pathogens prior to entering or recirculating through an air circulation system, which solves the heat dissipation problems described above. An example non-limiting system uses a finned heat sink to dissipate heat generated by UVC LEDs and direct or redirect airflow to provide more effective sterilization or deactivation of airborne pathogens.

### BRIEF DESCRIPTION OF THE DRAWINGS

FIG. 1 shows an example non-limiting embodiment of an air handler/recirculation system, including an airflow sanitizer.

FIG. 2 shows an example automotive use case for the airflow sanitizer.

FIG. 3 shows an example HVAC baffle box.

FIG. 4 is an elevated side perspective view of an example active UV filter structure.

FIG. 5 is a plan view of an example active UV filter structure.

FIG. 6 is a schematic view of the FIG. 5 example UV filter structure.

FIG. 7 shows an example heat sink fin configuration.

FIG. 8 is an image of both horizontal and vertical adjustments to the heatsink fins.

FIG. 9 shows an example test apparatus.

FIGS. 10A & 10B show example test results.

FIGS. 11A & 11B show example test prototypes.

FIG. 12 shows an example control system.

### DETAILED DESCRIPTION OF EXAMPLE NON-LIMITING EMBODIMENTS

One embodiment provides a germicidal UVC LED array that includes a fin design that uses a heatsink to dissipate the high-powered UVC LED-generated heat, redirect the air to increase or decrease the air pressure, providing pressure stabilization, and redirect the air to a channel to expose it to the maximum amount of irradiation from the UVC LEDs. This design creates air vortexes or vortices to expose pathogens to radiation for a more extended period of time and/or direct the pathogens to higher light intensity regions illuminated by the LEDs. The fin arrangement with the heatsink can be arranged at any angle. It can be placed around the edge of the duct system to provide a frame. An example non-limiting system thus provides sterilization or sanitizing to deactivate airborne pathogens carried by an air flow.

In one embodiment, the heat sink fins are precisely aligned to redirect and guide airflow directly across the UVC LED array to optimize ultraviolet irradiation of the moving air. This alignment is achieved by adjusting the fins along the X, Y, and Z axes, enabling multi-dimensional pivoting to establish ideal angular orientations. By dynamically positioning the fins to maximize airflow exposure to the UVC light field, the system significantly increases irradiation dosage, dwell time, and overall disinfection efficiency.

An example design includes an air filter frame, heat sink fins uniquely designed with ultraviolet (UV) light emitting diode (LEDs) disinfection irradiance and a power module that provides power to each heat sink fin. The air sterilizing system uses a novel frame design that encapsulates any size air filter and the novel frame integration with adjustable fins with UVC LEDs. The system provides the utility of an air cleaning chamber, adapted to support HVAC filters. The system employs a unique array of UVC light radiation in an intake chamber and return Air Duct (filter side 1) and on an outlet side of chamber (filter side 2).

Each air cleaning apparatus includes sterilization UV light exposing intake air, outlet and air filter surface area where the air is drawn through a filter that is irradiated with a UVC light energy comprised of a UV germicidal air disinfection system. The mechanism eliminates pathogens such as bacteria, mold, mildew, allergens, and deactivates viruses such as SARS CoV-2,

The technology herein further provides an effective method to eliminate the activation of airborne pathogens prior to entering the air circulation system.

In more detail, one embodiment provides an air filter encapsulation with integrated sensors using UVC LEDs emitting at wavelength of 200-280 nanometers range to sterilize and deactivate airborne pathogens, on the surfaces of a conventional filter and in an air intake and outlet. In clinical testing performed by a third party laboratory using active aerosolized SARS-CoV-2, embodiments described herein reached over 99% efficacy for wavelengths between 275 and 280 nanometers, and reached over 98% efficacy using wavelengths between 265-275 nanometers. Each design includes an air filter frame, and heat sink fins that are uniquely designed to cool ultraviolet (UV) light emitting diode (LEDs) disinfection arrays while directing or redirecting air flow so as to increase or at least not interfere with flow in close proximity to or over the LEDs.

The air sterilizing system uses a novel frame design that encapsulates any size air filter and the novel frame integration design of arrayed UVC LEDs. The example embodiments thus provide utility of air cleaning encapsulation, adapted to support HVAC filters. The system employs a unique array of UVC lamps in intake chamber and return Air Duct (filter side 1) and on an outlet side of chamber (filter side 2).

A power control module provides controlled energy to UVC heat sink fins to illuminate UV light at a given power; each UVC enabled heat sink fin is powered independently and has integrated controller for power management.

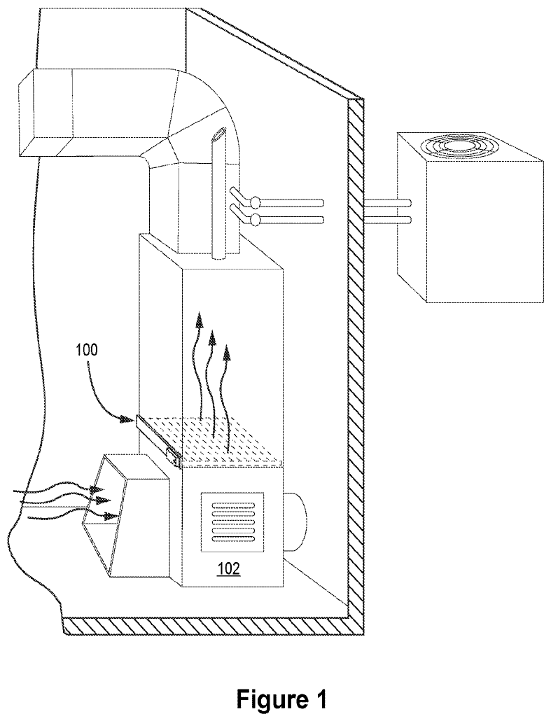

FIG. 1 shows an example air handling/recirculation system, including a modular air filter encapsulation system 100 as described above. The FIG. 1 system can comprise a conventional HVAC system including an inlet air vent or venting system (bottom), a conventional blower 102 (to pull air in through the inlet air vent and propel it through the HVAC system), a heat exchanger (which may add heat to and/or remove heat from the propelled air flow in a conventional fashion), and an outlet air vent (top) that delivers air to one or more outlet vents. The FIG. 1 system further includes a modular air filter encapsulation system 100 placed in the path of the air flow (either before or after the blower, depending on the HVAC system) such that all air that recirculates through the air handling/recirculating system must pass through the encapsulation system 100.

The modular air filter encapsulation system 100 in this embodiment includes a conventional air filter element that entraps small airborne particles (dust, droplets, aerosols, etc.) to prevent them from recirculating through the system. However, other arrangements may or may not include such a conventional particulate air filter or may or may not be integrated with such a conventional particulate air filter. For example, some embodiments may include UV illumination using LEDs that are integrated with the particular air filter, while other embodiments may provide a structure separate or separable from a conventional particulate air filter to provide such UV-LED illumination.

In the particular example embodiment shown in FIG. 1, a UV-C germicidal LED illumination system is provided on a frame or housing that holds, surrounds and/or encapsulates the conventional air filter element. The illumination system is configured to irradiate one or both sides of the undulating surfaces of the conventional air filter element and/or inflow air into the filter element and/or outflow air out of the filter element. The illumination system provides sufficient intensity of germicidal ultraviolet light to kill pathogens such as bacteria and viruses.

FIG. 1 shows the frame or housing and associated filter element in a horizontal orientation within the air handler. However, the frame or housing and associated filter element could be oriented vertically, or in any other orientation. Similarly, the FIG. 1 example shows a planar rectangular frame or housing or associated filter element, but other embodiments can have any desired shape such as non-planar, three-dimensional, circular, ellipsoid, pentagonal, octagonal, or shaped in any multi-sided shape. The particular shape, structure and size of the filter or housing and associated filter element will in general depend on the particular application such as the particular air handler.

Example spacing/dimensions of the UVC arrays may be to accommodate conventional disposable or non-disposable/reusable or non-reusable residential, commercial, industrial or other air filter elements such as 10″×10″×1″, 12″×12″×1, 12″×12″×2,″ 14″×20″×1″, 14″×20″×2″, 15″×20″×1, 15″×20″×2″, 15″×20″×3″, or any other standard or non-standard filter element in any shape, size, dimensions and materials. Some example frames may accommodate filter elements that are non-planar and/or non-rectangular such as cabin air filters of various different configurations, filter sheets or rolls, or other filter arrangements or configurations. Example frames may accommodate filter elements with any maximum efficiency reporting value (MERV) ratings such as MERV 8 to 13.

There are several different designs for conventional particulate filters, including (see e.g., “Filters 101: HVAC Filtration Options” retrieved from modernize.com/homeowner-resources/hvac/hvac-filtration-options-filters-101):
Flat-Paneled Fiberglass Filters: These disposable filters contain layered fiberglass and a metal reinforcing grate to prevent collapsing often surrounded by a cardboard frame. While the inexpensive filters protect HVAC components, they offer little by itself when it comes to air purification but the addition of UV illumination as described herein can make a big improvement.
Pleated Media Filter: These filters are disposable and carry a MERV rating of between 5 and 13 with high-efficiency versions rating between 14 and 16. The pleats are designed to increase the surface area of the filter, which in turn, raises its filtration efficiency. While a pleated filter falls short in its air filtration efficiency when compared to HEPA filter below, they provide less airflow resistance and are effective at suppressing fan noise at a lower pricing point.
HEPA Filter: HEPA filters provide the highest level of protection against air borne particles according to the Occupational Safety and Health Administration as well as the Environmental Protection Agency. The filters are capable of trapping particles as small as 0.3 microns or larger and catch 99.97-percent of air borne particles. HEPA air filters carry a MERV rating of between 17 and 20. It should be noted that many residential systems cannot support a HEPA filter due to its size and airflow restrictions without modification from an HVAC contractor but that this situation in changing. Meanwhile, HEPA filters are used in many commercial settings such as office buildings and the benefits of adding UV illumination as described herein can thus be used to protect a larger number of people at once.
Reusable Air Filters: Another HVAC filter option is a washable or reusable air filter. While this type of filter is more expensive than its disposable counterpart, it can be reused after a simple cleaning. Drawbacks include a low MERV rating of between 1 and 4 and the chances of attracting mold and/or mildew growth from installing a still damp filter. Adding UV illumination as described herein can help dry a damp filter as well as provide additional protection against pathogens.

Example embodiments can incorporate UV LEDs into each or any of such (or other) conventional particulate filter designs.

The provided UV arrays 108, 110 are arranged and spaced so that all filter element 124 surfaces are illuminated and ingress and egress air flow is also illuminated for sufficient time with sufficient intensity to destroy or deactivate pathogens.

As air moves through an HVAC system, air filters trap and collect large and small particles such as dust, allergens and microorganisms. According to the American Society of Heating, Refrigerating and Air-Conditioning Engineers (ASHRAE), this filtration helps provide healthier indoor air quality. A MERV rating of ≥13 (or ISO equivalent) is efficient at capturing airborne viruses, and MERV 14 (or ISO equivalent) filters are preferred. High efficiency particulate air (HEPA) filters as noted above are more efficient than MERV 16 filters. Generally, particles with an aerodynamic diameter around 0.3 μm are most penetrating; and efficiency increases above and below this particle size. Overall effectiveness of reducing particle concentrations depends on several factors such as:
Filter efficiency
Airflow rate through the filter
Size of the particles
Location of the filter in the HVAC system or room air cleaner.

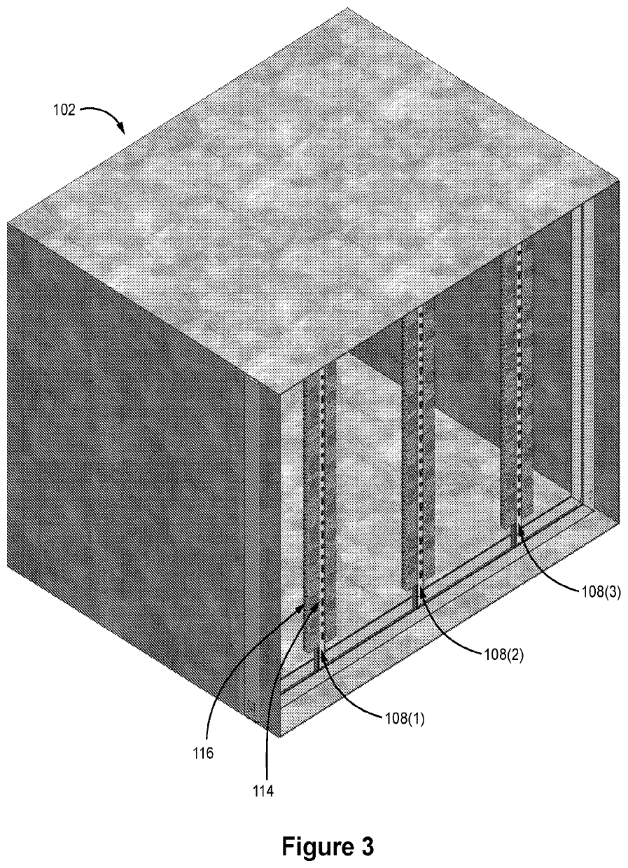

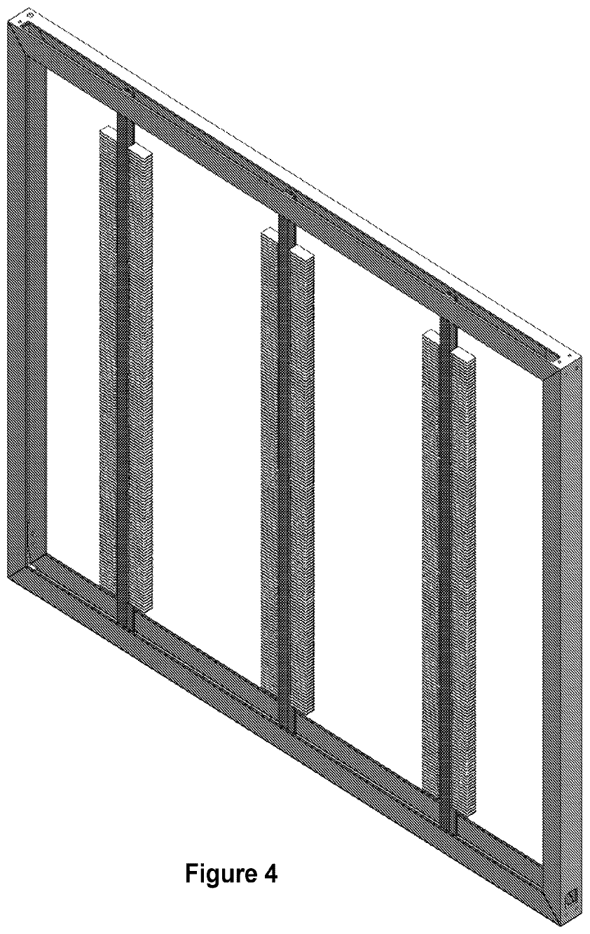

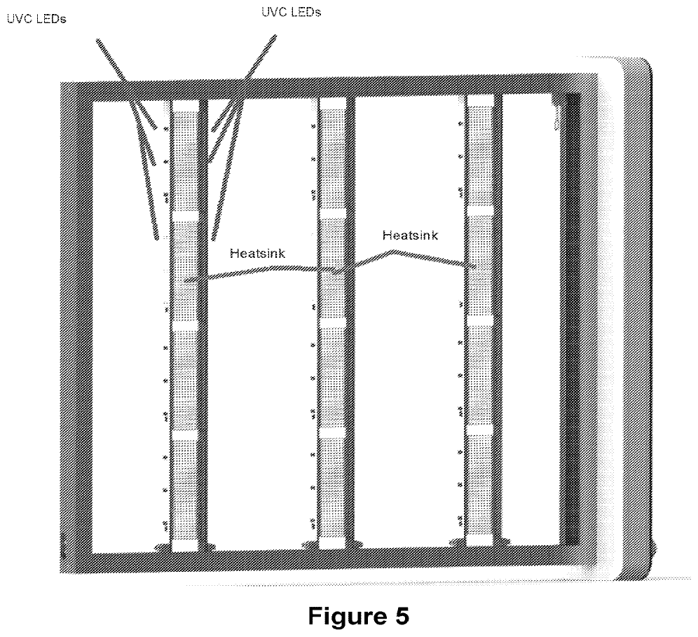

FIG. 3 shows an example filter housing, air baffle box or other air carrying structure 102 through which air flows. Structure 102 may be the filter housing as shown in FIG. 1, or it could be a different baffle or other air housing somewhere else in the system. In example embodiments herein, the housing 102 supports one or more ultraviolet (UV) light emitting arrays 108. In one embodiment, such UV light emitting arrays 108 are evenly or unevenly distributed across the surface of a replaceable filter element (now shown) to provide sufficient UV illumination on the surface(s) of the filter and in ingress and egress air flows. In many embodiments, the filter element is corrugated or undulating to increase filter surface area, creating a meandering or undulating filter surface topography. The UV illumination arrays 108 are in one embodiment disposed and spaced above and across the surface of the filter element in such a way as to illuminate every part of such meandering/undulating filter surfaces as well as air flowing toward, away and/or through the filter element.

In some embodiments, UV arrays 108 are respectively disposed on the egress side of the filter element. In some embodiments, UV arrays 108 may be disposed only on the ingress side of the filter element or UV arrays 108 may be disposed on both an ingress side and an egress side of the filter element. In some embodiments, the UV arrays 108 are structured to illuminate the filter surface(s) as well as egress air flow but in some embodiments the UV arrays may illuminate only the air flow without illuminating the filter surface. In some embodiments, the UV arrays 108 may contact or be integrated directly into the filter element instead of being spaced apart and above the filter element.

In the example shown, each UV array 108 may comprise a longitudinal strip of conductive or non-conductive material that bears a plurality of light emitting diode illuminators 114 and associated electrical and/or data conductors. The number of UV illuminators 114 and their arrangements and spacings may depend on a number of factors including the filter element size, the type of filter element, the air flow rate, the degree of germicidal protection needed, the power and field of view of illuminators 114, and other factors. Similarly, the number of arrays 108 may depend on various factors including the size and shape of the filter element, the air flow rate, the degree of germicidal protection, the power and field of view of illuminators 114, and other factors. The arrays 108 may be in registry to one another, offset from one another, or have no positional correspondence with respect to one another. In the embodiment shown the arrays 108 are parallel to one another, but in other embodiments the arrays 108 can be oriented at right angles to the arrays 110, or may be oriented at any desired orientation relative to one another. The arrays 108 in the embodiment shown are at right angles to edges of housing 102, but in other embodiments the arrays may be oriented at any angle relative to the edges of the housing 102. In the embodiment shown all arrays 108 are parallel to one another, but in some embodiments arrays 108 can have different orientations relative to one another. In the embodiment shown arrays 108 are coplanar so they are equidistant from the filter surface(s), but in other embodiments arrays 108 may lie in different planes to provide different distances between the filter element and the arrays and/or to accommodate non-planar filter elements.

As can be seen in FIG. 3, the UV arrays 108 in one embodiment each comprise a thin strip of flexible or rigid material surrounded on each of two sides by stacked finned heat sink structures 116. In the example embodiment, these stacked finned heat sink structures comprises arrays of thin metallic heat-conductive plates or fins such as aluminum for example of the general type used by automotive radiators or slant/fin baseboard heaters found in some homes. The fins or plates in example embodiments may be highly reflective such as shiny metallic or shiny metallic coated plastic. Each heat-conductive plate or fin is thermally coupled to a substrate bearing the UVC LED(s) 114 so that heat produced by the LED(s) is conducted away from the LED(s) and is radiated and dissipated by the plates or fins. A predetermined spacing and/or angle is provided between adjacent fins in the stacked structures so that air can flow between and through the fins to carry heat away from the fins. The angle and orientation of the heatsink fins compensate for increased static air pressure introduced by the heatsink to normalize airflow velocity.

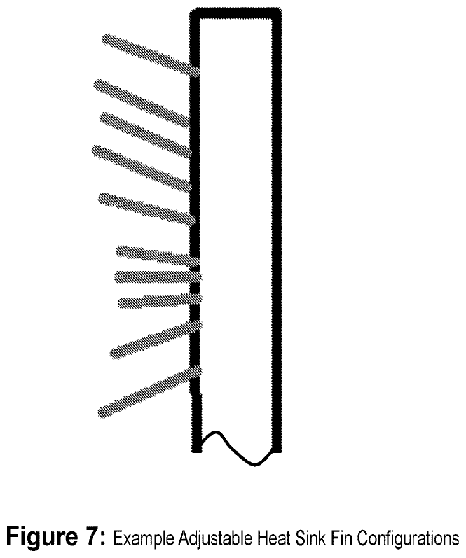

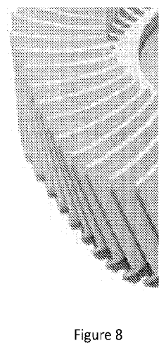

In one example embodiments, the fins of the stacked fin structures 116 are fixed in position so that all of the fins are fixedly attached to the support columns. The fins shown may be parallel to one another and may have a horizontal orientation. However, in other embodiments, the fins may be parallel to one another and have an orientation other than horizontal, for example 2 degrees from horizontal or 5 degrees from horizontal or 10 degrees from horizontal or 15 degrees from horizontal or 20 degrees from horizontal or 25 degrees from horizontal or 30 degrees from horizontal or 35 degrees from horizontal or 40 degrees from horizontal or some other angle from horizontal. For example, a heatsink could have a grid shape to dissipate heat and remove swirls from the airflow. Or we could use heatsinks that are angled symmetrically to channel the air in the optimal path of the LEDs. In one embodiment, we can rotate the heatsink fins both from the horizontal and vertical sides. See FIG. 8. You can for example have a 10 degrees horizontal and 15 degrees vertical. The angle adjustments can be independent, meaning the top half can start with a 10 degree angle, but as it gets to the center point the fins will have a 0 degree angle and then may start on a path toward a negative 10 angle. See FIG. 7.

In other embodiments, the fins or plates may have adjustable orientations relative to the columns. For example, the fins may have structures such as adjustable louvred shutters or automobile adjustable vents, that allows a user or installer to adjust (e.g., by moving a rod, wire or other control) all fins in a stack such that the fins remain parallel to one another but have common orientations that can be adjusted in a range such as from 0 degrees from horizontal to ±5 degrees from horizontal or from 0 degrees from horizontal to ±10 degrees from horizontal or from 0 degrees from horizontal to ±20 degrees from horizontal or from 0 degrees from horizontal to ±30 degrees from horizontal or from 0 degrees from horizontal to ±45 degrees from horizontal or from 0 degrees from horizontal to ±60 degrees from horizontal or from 0 degrees from horizontal to ±some other angle from horizontal. Or in some embodiments the fin orientations may be adjusted in only one direction relative to horizontal. In one embodiment, the fins are oriented in fixed positions exposing thin profiles to an oncoming air flow that minimize obstruction to oncoming air flow in a direction normal to the plane of the panel while nevertheless radiating heat generated by the LED arrays into the airflow so the airflow can carry the heat away.

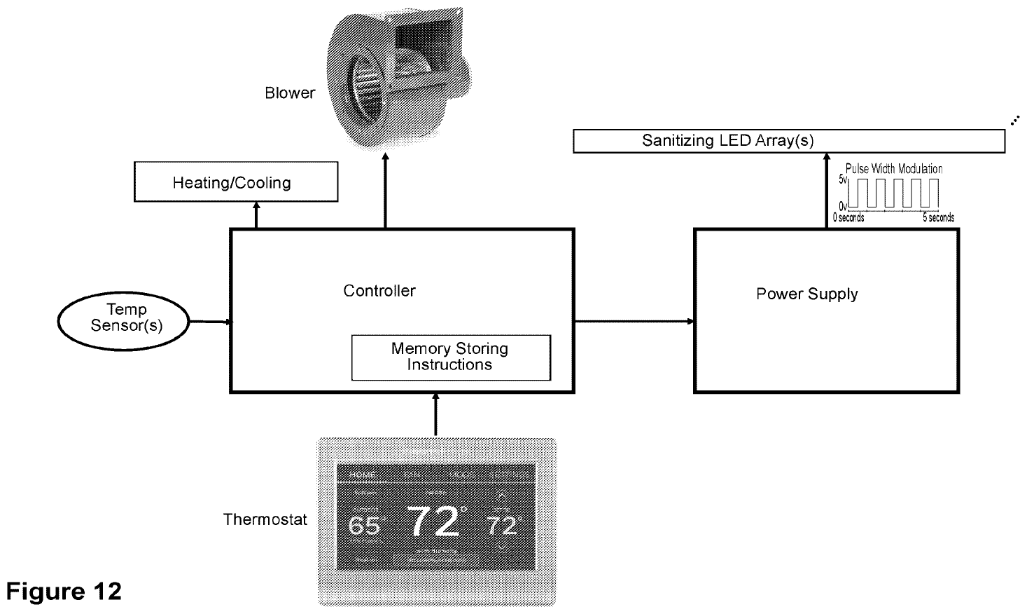

The rotation of each fin 106, 120 is designed specifically for maximum irradiance and exposure of photons received by surface area as UVC LEDs are illuminated, and protection of eye or skin of human and/or to control and divert the direction of air flowing through the fin stacks. The direction of each fin 106, 120 is designed (e.g., sized) based on airflow and filter size. In one embodiment, each fin rotates at an appropriate angle, designed for maximum exposure/irradiance and preventing shadowing effects. In one embodiment, the fins are oriented to create laminar air flows between the plates that eliminate vortices and disturbances. In other embodiments, the fins are oriented to intentionally create microvortices close to the maximum irradiance volumes of the LEDs to thereby increase the time pathogens carried by the air whirl about in close proximity to the LEDs, thereby increasing the effectiveness of high-intensity UVC radiation the LEDs emit in deactivating pathogens the air carries. In one embodiment, the HVAC air handler controller may turn the LEDs on by supplying current to them whenever the HVAC air handler fan is on, and my turn the LEDs off by discontinuing the supply of current to them whenever the HVAC air handler fan is off. In one embodiment, the HVAC air handler controller may activate the LEDs during both cooling and heating operations. To prevent overheating, it is possible to control the intensity and/or the duty cycle of the LEDs based on monitored temperature of the duct and/or monitored temperature of the frame. See FIG. 12 controller that may execute instructions stored in non-transitory memory to perform the above as well as to control the HVAC blower and heating/cooling equipments in response to manual and/or automatic commands received wirelessly or over a wire(s) from a thermostat or other user interface.

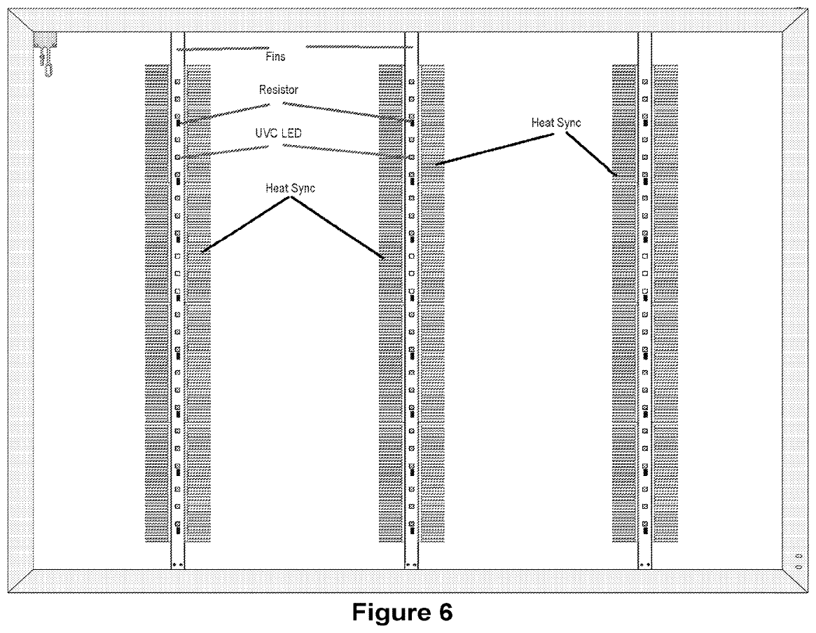

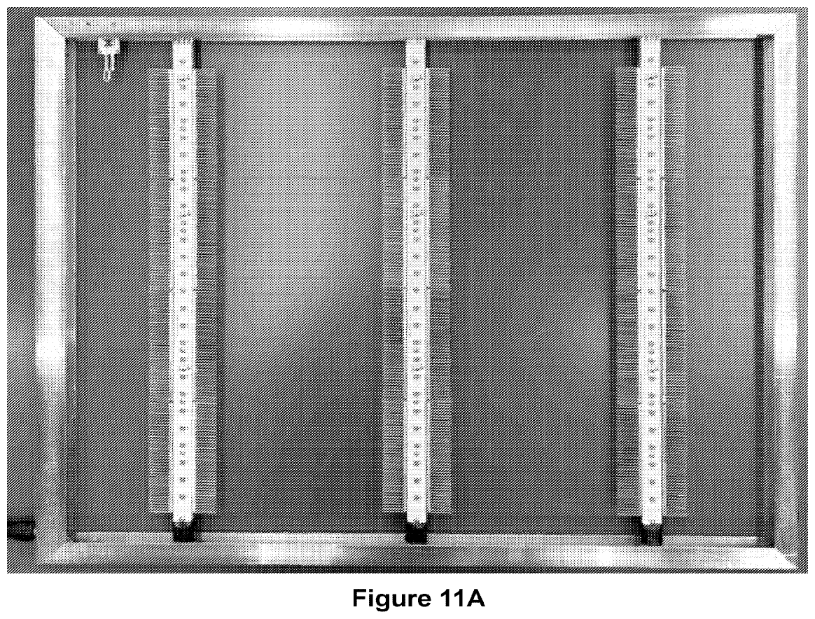

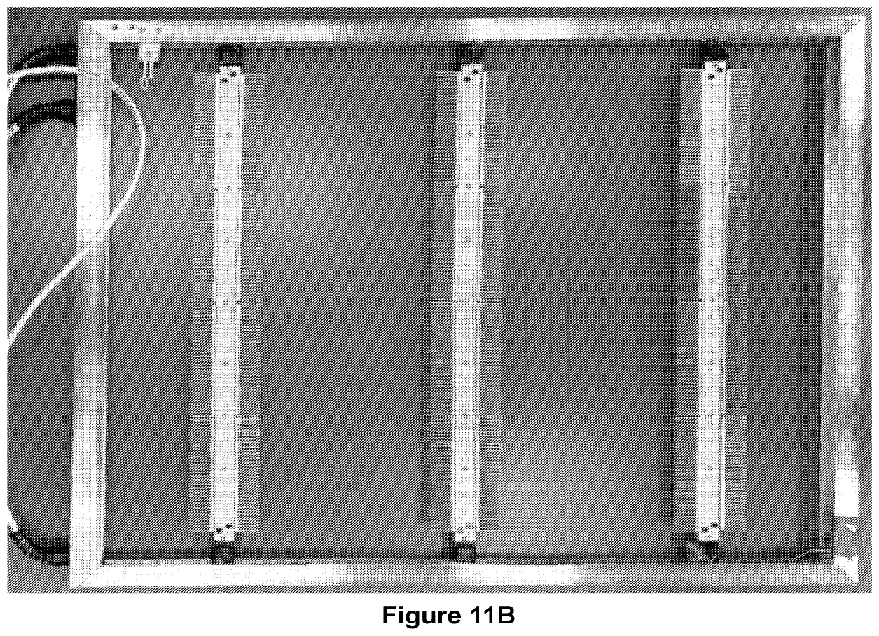

FIG. 6 shows that in one embodiment, the UV LEDs may be evenly spaced along the array. For example, FIG. 6 shows three arrays each having 24 evenly-spaced LEDs. Other embodiments may have different numbers of arrays, different numbers of LEDs on each array, and/or different spacings between LEDs. For example, the embodiment shown in FIG. 11A has 30 or more LEDs in each array, with the LEDs being unevenly spaced. In the FIG. 11A design, certain of the LEDs are defined by groups of three LEDs that are closely spaced to one another, providing non-uniform light intensity across the plane of the panel with some portions having higher light intensity than other portions but all areas of the plane illuminated by at least a minimum light intensity. In the examples shown, all LEDs are aimed in the same direction (e.g., in a direction facing the oncoming air stream) but other aiming directions and/or orientations are possible.

While the disclosed arrangement uses linear LED arrays for convenience in order to provide linear stacks of spaced-apart heat sinks along the axis of each array, other arrangements are possible. For example, one embodiment may scatter the LEDs across an air-permeable plane or volume and thermally couple each LED or each group of LEDs to one or more heat sink fins configured to remove heat from the LED(s).

Our example system allows exposure of 6000 Joules/m2 for eliminating colonies of 6000-10,000 RNA viruses. As such, UVC LEDs are exposed at range of a few seconds to 60 an hour with closely spaced UVC LED arrays in vertical and horizontal direction. Our system allows logarithmic reduction of 1,000-10,000 colony forming units (CFUs).

### Further Use Case

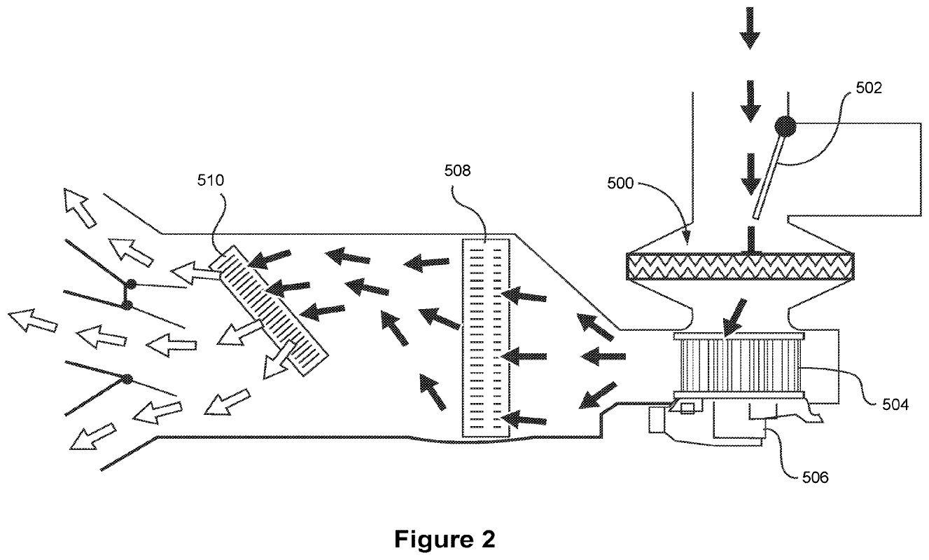

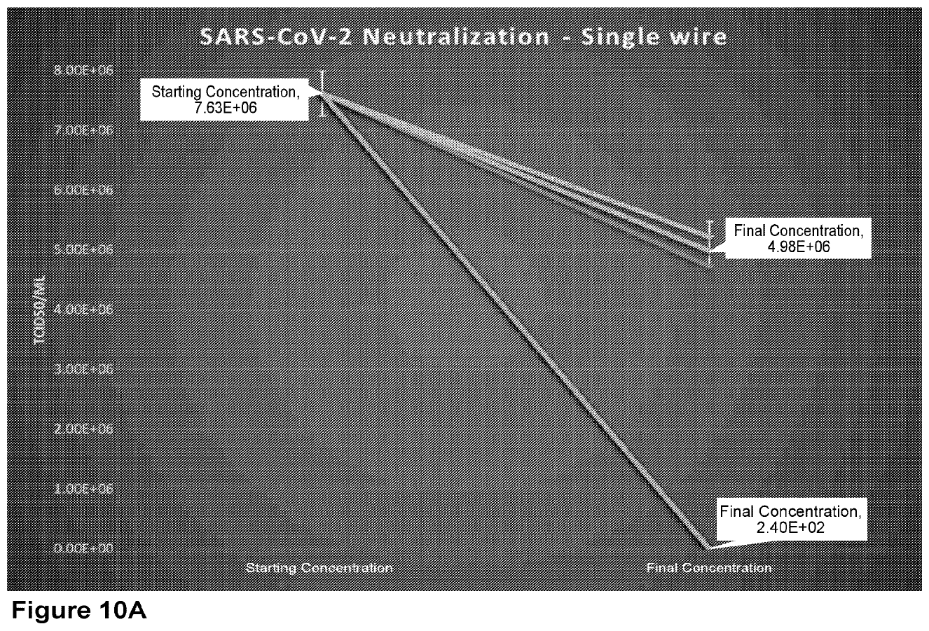

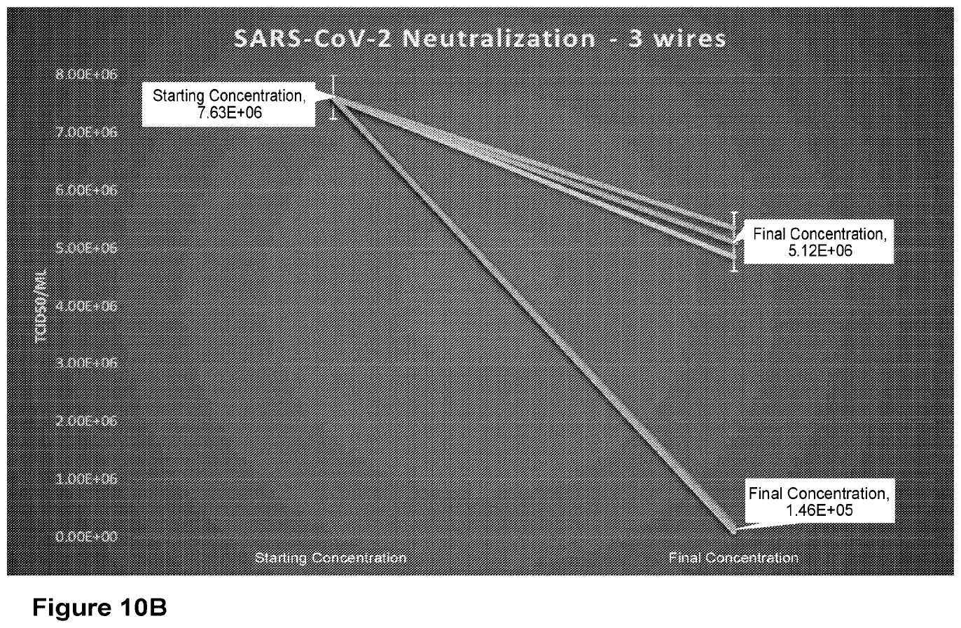

FIG. 2 shows an additional example use case for the disclosed encapsulated filter. FIG. 10 shows use, installation and placement of the encapsulated filter as a cabin air filter in a vehicle air recirculation system. In this example, recirculating air through a shutter or vent 502 flows through the UV encapsulated filter 500 under the force of blower 504, which pushes the air through a heat exchanger 508 and a diverter 510 to output vents on a dashboard, cockpit or other parts of the vehicle. Other use cases such as mask filter elements, ventilator filter elements, vacuum cleaner filter elements, household fans or blowers, humidifiers or dehumidifiers, or any other device that moves or flows air that may be breathed by humans or animals, may benefit from the application of the technology described herein.

### EXAMPLE 1

One embodiment of air filter encapsulation system is comprised of uniquely designed:
Air filter slot encapsulation system
UVC LED arrays
fin angle adjusters or fixed fin angle mounting
Senor Module (see FIG. 12)
Power Module (See FIG. 12).

Array placement, orientation and power design of UVC array maximize air filter surface and intake and outlet with irradiance (radiant power received by surface) of 2000-8000 micro-watt/cm2 with fluence (UV exposure dose rate of 10-80 Joules/m2/Sec.An array of UVC Germicidal Disinfection LED is used to eliminate pathogens such as bacteria, mold, mildew, allergens, and deactivate viruses such as SARS CoV-2.

### EXAMPLE II

The LEDs emit ultraviolet light in the 200-280 nanometers range to sterilize and deactivate airborne pathogens, on the surfaces of a conventional filter and in an air intake and outlet.

### EXAMPLE III

In clinical testing performed by a third party laboratory using active aerosolized SARS-CoV-2, embodiments described herein reached over 99% efficacy for wavelengths between 275 and 280 nanometers. For this test, the frame shown in FIG. 11A (and as described above) was populated with UV LEDs of Nichia part number (NCSU434C) having a data sheet found at led-ld.nichia.co.jp/api/data/spec/led/NCSU434C(T)-E(6724C).pdf. Peak wavelength was a minimum of 275 nm and a maximum of 285 nm and was centered around 280 nm (spectrum half width=10 nm). Peak wavelength tolerance of the devices was plus or minus 1.8 nm. The LEDs were driven at 350-500 ma of forward current (maximum rating=500 ma), providing a radiant flux value (specification) of 0.110 mW. One common wire was used in this embodiment to supply current to the three different LED arrays.

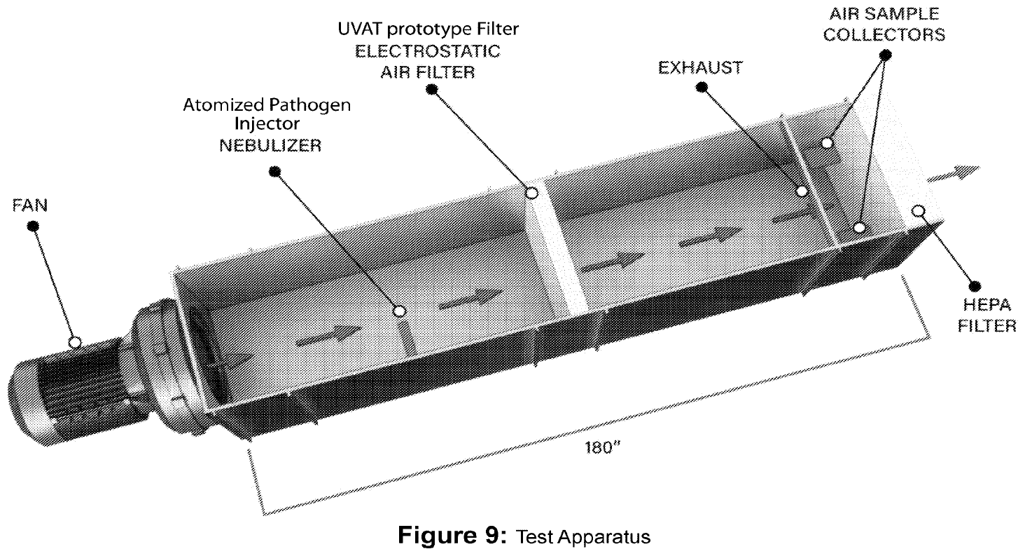

Testing was conducted in a modified HVAC system constructed to comply with modified ANSI/ASHRAE standards. The system was placed inside a sealed, temperature-controlled 20′×11′×9′ chamber (as shown schematically in FIG. 9) following Biosafety Level 3 (BSL-3) standards. Airflow was generated upstream by a SystemAir Prio 315 axial circular duct fan (S/N: 93267/1003679708-029/20180529) measured at approximately 1 m/s with a ±3% tolerance across the ducting. The air traveled through ducting where nebulization of pathogen occurs and flowed through the UV air filter prototype with the UV lights facing on the downstream side. After passing the UV air filter prototype, the air flowed into a boxed duct, where air sampling occurred using two sampling probes, each connected to a vacuum device. The temperature during all test runs was approximately 70±2° F. with a relative humidity of 47%. For all tests, fan speed was set at approximately 6 m/s±3% across the ducting. Air sampling collection occurred for 15 minutes per challenge for each test. The air sample collection volumes were set to 15-minute continual draws at the point of sampling. Three controls and three air passes were conducted using the same methodology for each test.

FIG. 10A shows aerosolized SARS-CoV-2 neutralization after a single air pass using the UV LED air filter—single wire for wavelengths between 275 and 280 nanometers. The results plotted in FIG. 10 display recovered active SARS-CoV-2 with and without the 280 nm UV LED air filter during testing. The controls showed a natural loss of aerosolized SARS-CoV-2 after a single air pass under controlled conditions without the test device. Across three air passes against SARS-CoV-2, the module lowered a starting concentration of 7.63×10⁶ TCID50/mL to 4.80×10², 1.20×10², and 1.20×10² TCID50/mL, averaging to approximately 2.40×10² TCID50/mL after a single air pass.

Here are test results Data and Calculated Percentage Reductions for SARS-CoV-2 using the UV filter—single wire prototype for wavelengths between 275 and 280 nanometers:

| | Starting | Final |
|---|---|---|
| | Concentration | Concentration |
| Control 1 (TCID50/mL) | 7.63E+06 | 4.99E+06 |
| Control 2 (TCID50/mL) | 7.63E+06 | 5.23E+06 |
| Control 3 (TCID50/mL) | 7.63E+06 | 4.73E+06 |
| Average Control (TCID50/mL) | 7.63E+06 | 4.98E+06 |
| Average % Gross Reduction - | | −34.66 |
| Control | | |
| Experiment 1 (TCID50/mL) | 7.63E+06 | 4.80E+02 |
| Experiment 2 (TCID50/mL) | 7.63E+06 | 1.20E+02 |
| Experiment 3 (TCID50/mL) | 7.63E+06 | 1.20E+02 |
| Average Experiment | 7.63E+06 | 2.40E+02 |
| (TCID50/mL) | | |
| Average % Gross Reduction - | | −99.997 |
| Experiment | | |
| % Net Reduction | | −99.995% |

***the value of 1.2E+02 indicates a titer that is lower than the specified limit of quantitation.

### EXAMPLE IV

In clinical testing performed by a third party laboratory using active aerosolized SARS-CoV-2, embodiments described herein reached over 98% efficacy using wavelengths between 265-275 nanometers.

The frame for this test (shown in FIG. 11B) was populated with UV LEDs Crystal IS part number KL265-50W-SM-WD (engineering samples). The data sheet may be found at store.cisuvc.com/datasheets/crystal-is/cis_klaran_wd_ds_031021.pdf These LED were driven at 50V (output>70 mW). Three wires were used to supply current to the three different LED arrays as shown.

As above, testing was conducted in a modified HVAC system constructed to comply with modified ANSI/ASHRAE standards. The system was placed inside a sealed, temperature-controlled 20′×11′×9′ chamber (as shown in FIG. 9) following Biosafety Level 3 (BSL-3) standards. Airflow was generated upstream by a SystemAir Prio 315 axial circular duct fan (S/N: 93267/1003679708-029/20180529) measured at approximately 1 m/s with a ±3% tolerance across the ducting. The air travels through ducting where nebulization of pathogen occurs and flows through the UV air filter prototype with the UV lights facing on the downstream side. After passing the UV air filter prototype, the air flowed into a boxed duct, where air sampling occurred using two sampling probes, each connected to a vacuum device. The temperature during all test runs was approximately 70±2° F. with a relative humidity of 47%. For all tests, fan speed was set at approximately 6 m/s±3% across the ducting. Air sampling collection occurred for 15 minutes per challenge for each test. 3. The air sample collection volumes were set to 15-minute continual draws at the point of sampling. Three controls and three air passes were conducted using the same methodology for each test.

The results plotted in FIG. 10B display recovered active SARS-CoV-2 with and without placement of a 265 nm UV LED air filter as shown in Figure ______ herein during testing. The controls showed a natural loss of aerosolized SARS-CoV-2 after a single air pass under controlled conditions. Across three air passes against the SARS-CoV-2, the module lowered a starting concentration of 7.63×10⁶ TCID50/mL to 1.26×10⁵, 9.57×10⁴, and 2.17×10⁵ TCID50/mL, averaging to approximately 1.46×10⁵ TCID50/mL after a single air pass.

Here are test results Data and Calculated Percentage Reductions for SARS-CoV-2 using the UV air filter—3 wired prototype using wavelengths between 265-275 nanometers:

| | Starting | Final |
|---|---|---|
| | Concentration | Concentration |
| Control 1 (TCID50/mL) | 7.63E+06 | 4.85E+06 |
| Control 2 (TCID50/mL) | 7.63E+06 | 5.36E+06 |
| Control 3 (TCID50/mL) | 7.63E+06 | 5.15E+06 |
| Average Control (TCID50/mL) | 7.63E+06 | 5.12E+06 |
| Average % Gross Reduction - | | −32.85 |
| Control | | |
| Experiment 1 (TCID50/mL) | 7.63E+06 | 1.26E+05 |
| Experiment 2 (TCID50/mL) | 7.63E+06 | 9.57E+04 |
| Experiment 3 (TCID50/mL) | 7.63E+06 | 2.17E+05 |
| Average Experiment | 7.63E+06 | 1.46E+05 |
| (TCID50/mL) | | |
| Average % Gross Reduction - | | −98.08 |
| Experiment | | |
| % Net Reduction | | −97.14% |

The UV air filter prototypes described herein thus demonstrated the ability to reduce aerosolized SARS-CoV-2 in a controlled environment. The UV filter—single wire prototype for wavelengths between 275 and 280 nanometers observed an average 99.997% gross reduction of SARS-CoV-2 compared to a 34.66% viability loss after a single air pass. Testing the UV filter—3 wires using wavelengths between 265-275, the device displayed a 98.08% gross reduction of SARS-CoV-2 compared to a 32.85% viability loss after an air pass.

When aerosolizing pathogens and collecting said pathogens, some variables cannot be fully accounted for, namely, placement of pathogen, collection volume, collection points, drop rate, surface saturation, virus destruction on collection, virus destruction on aerosolization, and possibly others. However, considering the variables, the UV American air filters achieved a measurable reduction after a single air pass. The decline of recoverable active SARS-CoV-2 was consistent with asserting that the device can decrease the concentration of active pathogens in the air. Overall, under controlled conditions, the prototype displayed an average 4.50 gross log reduction against SARS-CoV-2 after a single air pass with the single-wired UV air filter for wavelengths between 275 and 280 nanometers and a 1.72 gross log reduction of SARS-CoV-2 with the three-wired UV air filter using wavelengths between 265-275 nanometers.

### EXAMPLE V

The frames are populated with Klaran® WD Series UVC LED with a peak wavelength of 260 nm-270 nm providing a minimum of 80 mW of output. Based on the above, the expected reduction of the pathogens described above is 99.999%.

### EXAMPLE VI

The LEDs may emit pathogen-killing ultraviolet light in the range of 200-205 nm or 205-210 nm or 210-215 nm or 215-220 nm or 220-225 nm or 225-230 nm or 230-235 nm or 235-240 nm or 240-245 nm or 245-250 nm or 250-255 nm or 255-260 nm or 260-265 nm or 265-270 nm or 270-275 nm or 275-280 nm.

UV-LEDs tend to be narrow band sources and are typically described and identified by their most dominant spectral output. If measured with a spectral radiometer, the user would see that the manufacturer has binned the individual LED chips so that the most intense UV output is clustered around the dominant name line. In reality, it is common for the actual spectral emission of a UV-LED to extend plus/minus 8-15 nm in either direction at the half-maximum power point from the maximum. See e.g., Raymont et al, “Measuring the Output of UV LEDs” RadTech UV & EB Technical Conference Proceedings (2010).

All patents and publications cited herein are incorporated herein by reference for all purposes.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

1. A germicidal structure comprising:
an ultraviolet light array comprising ultraviolet light emitting elements provide ultraviolet light intensities of 6000 Joules/m² for deactivating colonies of 6000-10,000 RNA viruses to enable logarithmic reduction of 1,000-10,000 colony forming units (CFUs); and
heat conductive fins thermally coupled to the ultraviolet light emitting elements,
wherein the fins dissipate heat generated by the ultraviolet light emitting elements and are structured to direct or divert air flow to increase the germicidal efficacy of the ultraviolet light intensities.

2. The germicidal structure of claim 1 wherein the structure reduces aerosolized SARS-CoV-2 by over 95% in a single pass.

3. The germicidal structure of claim 1 wherein the structure reduces aerosolized SARS-CoV-2 by over 98% in a single pass.

4. The germicidal structure of claim 1 wherein the structure reduces aerosolized SARS-CoV-2 by over 99% in a single pass.

5. The germicidal structure of claim 1 wherein the structure reduces airborne pathogens in air flowing through the structure by over 95% in a single pass.

6. The germicidal structure of claim 1 wherein the structure reduces airborne pathogens in air flowing through the structure by over 98% in a single pass.

7. The germicidal structure of claim 1 wherein the structure reduces airborne pathogens in air flowing through the structure by over 99% in a single pass.

8. The germicidal structure of claim 1 wherein the ultraviolet light emitting elements emit ultraviolet light in the range of 200 to 280 nanometers, and preferably in the range of 265 to 280 nanometers or 265 to 275 nanometers or 275 to 280 nanometers.

9. An air filter comprising:
a first layer that captures airborne particulate matter, and
a second layer that emits ultraviolet light into an oncoming air stream, the second layer comprising at least one array of light emitting diodes thermally coupled to heat-conductive fins, the heat-conductive fins radiating heat generated by the light emitting diodes to prevent the light emitting diodes from overheating, the fins configured to allow air to pass by the at leasts one array without significant obstruction to air flow.

10. The air filter of claim 9 wherein the filter reduces aerosolized SARS-CoV-2 by over 95% in a single pass.

11. The air filter of claim 9 wherein the filter reduces aerosolized SARS-CoV-2 by over 98% in a single pass.

12. The air filter of claim 9 wherein the filter reduces aerosolized SARS-CoV-2 by over 99% in a single pass.

13. The air filter of claim 9 wherein the filter reduces airborne pathogens in air flowing through the structure by over 95% in a single pass.

14. The air filter of claim 9 wherein the filter reduces airborne pathogens in air flowing through the structure by over 98% in a single pass.

15. The air filter of claim 9 wherein the filter reduces airborne pathogens in air flowing through the structure by over 99% in a single pass.

16. The air filter of claim 9 wherein the ultraviolet light emitting elements emit ultraviolet light in the range of 200 to 280 nanometers, and preferably in the range of 265 to 280 nanometers or preferably in the range of 265 to 275 nanometers or preferably in the range of 275 to 280 nanometers.

17. The air filter of claim 9 wherein the air filter comprises three linear LED arrays disposed parallel to each other, each linear array having at least 20 LEDs disposed thereon.
